(19) United States
(12) Patent Application Publication
(10) Pub. No.: US 2020/0345325 A1
(43) Pub. Date: Nov. 5, 2020

(54) AUTOMATED PATH CORRECTION DURING MULTI-MODAL FUSION TARGETED BIOPSY

(71) Applicants: KONINKLIJKE PHILIPS N.V., EINDHOVEN (NL); The University of Bristish Columbia, Vancouver, Bristish Columbia (CA)

(72) Inventors: AMIR MOHAMMAD TAHMASEBI MARAGHOOSH, ARLINGTON, MA (US); PURANG ABOLMAESUMI, VANCOUVER, BRITISH COLUMBIA (CA); PARVIN MOUSAVI, KINGSTON, ON (CA)

(21) Appl. No.: 16/962,861

(22) PCT Filed: Jan. 7, 2019
(86) PCT No.: PCT/EP2019/050191

§ 371 Date: Jul. 17, 2020

Related U.S. Application Data
(60) Provisional application No. 62/619,277, filed on Jan. 19, 2018.

(51) Int. Cl. A61B 8/08; A61B 8/12; A61B 8/00; G06N 3/08; A61B 10/02

(52) U.S. Cl. CPC A61B 8/085; A61B 8/12; A61B 8/5246; A61B 2560/0487; A61B 8/5223; G06N 3/08; A61B 10/0241; A61B 8/461

(57) ABSTRACT

The present disclosure describes ultrasound imaging systems and methods configured to delineate sub-regions of bodily tissue within a target region and determine a biopsy path for sampling the tissue. Systems may include an ultrasound transducer configured to image a biopsy plane within a target region. A processor communicating with the transducer can obtain a time series of sequential data frames associated with echo signals acquired by the transducer and apply a neural network to the data frames. The neural network can determine spatial locations and identities of various tissue types in the data frames. A spatial distribution map labeling the coordinates of the tissue types identified within the target region can also be generated and displayed on a user interface. The processor can also receive user input, the neural network determines spatial locations and identities of a plurality of via the user interface, indicating a targeted biopsy sample to be collected, which can be used to determine a corrected biopsy path.

### TECHNICAL FIELD

The present disclosure pertains to ultrasound systems and methods for identifying distinct regions of cancerous tissue using a neural network and determining a customized biopsy path for sampling the tissue. Particular implementations further involve systems configured to generate a tissue distribution map that labels the distinct types and spatial locations of cancerous tissue present along a biopsy path during an ultrasound scan of the tissue.

### BACKGROUND

Prostate cancer is the most common type of cancer in men and the third leading cancer-related cause of mortality in the United States. Over 230,000 American men are diagnosed with prostate cancer annually, and close to 30,000 die of the disease. Transrectal ultrasound imaging (TRUS) has been used by urologists for imaging the prostate, guiding biopsies, and even treating cancerous tissue. The prostate has heterogeneous echogenicity, however, and cancerous tissue is not distinguishable from healthy tissue in ultrasound images. As a result, existing techniques have fused TRUS data with pre-operative data gathered via multi-parametric magnetic resonance imaging (mpMRI), which can identify cancerous tissue, to improve biopsy guidance based on the presence of cancerous tissue. To transform possible cancerous locations identified via mpMRI into specific TRUS-derived coordinates for biopsy targeting, image registration techniques can be used.

One of the challenges with mpMRI-TRUS fusion techniques is the misalignment of biopsy locations on real-time 2D TRUS images, which leads to suboptimal biopsy targeting. This is due to the fact that the alignment between mpMRI and TRUS data may be performed only once, following the initial TRUS sweep of the prostate. In the time between image registration and biopsy, typically in the order of tens of minutes, the prostate can move and/or deform from the initial state that the 3D TRUS sweep was acquired from. The transformation resulting from the registration of mpMRI-TRUS data may thus be inaccurate at the time the biopsy is performed. Accordingly, new systems capable of recognizing and spatially delineating discrete regions of cancerous tissue during a biopsy may be desirable.

### SUMMARY

The present disclosure describes ultrasound imaging systems and methods for identifying distinct types of bodily tissue present along a biopsy plane, including the spatial locations of each tissue type identified. Tissue types delineated by the disclosed systems may include various grades of cancerous tissue within an organ, such as a prostate gland, breast, liver, etc. Example systems may be implemented during a biopsy procedure, for example a transrectal biopsy of a prostate gland, which may involve acquiring a time series of sequential ultrasound data frames from the region targeted for biopsy. Example systems may apply a neural network trained to determine the identity and spatial coordinates of cancerous tissue. This information can be used to generate a tissue distribution map of the biopsy plane along which the ultrasound data was acquired. Based on the tissue distribution map, a corrected biopsy path may be determined. The corrected biopsy path can incorporate user input regarding the prioritization of certain tissue types for biopsy in view of clinical guidelines, individual preferences, feasibility constraints, and/or patient-specific diagnoses and treatment plans, just to name a few. In some embodiments, instructions for adjusting an ultrasound transducer or biopsy needle in the manner necessary to arrive at the corrected biopsy path may be generated and optionally displayed.

In accordance with some examples, an ultrasound imaging system may include an ultrasound transducer configured to acquire echo signals responsive to ultrasound pulses transmitted along a biopsy plane within a target region. At least one processor in communication with the ultrasound transducer may also be included. The processor can be configured to obtain a time series of sequential data frames associated with the echo signals and apply a neural network to the time series of sequential data frames. The neural network can determine spatial locations and identities of a plurality of tissue types in the sequential data frames. The processor, applying the neural network, can further generate a spatial distribution map to be displayed on a user interface in communication with the processor, the spatial distribution map labeling the coordinates of the plurality of tissue types identified within the target region. The processor can also receive a user input, via the user interface, indicating a targeted biopsy sample, and generate a corrected biopsy path based on the targeted biopsy sample.

In some examples, the time series of sequential data frames may embody radio frequency signals, B-mode signals, Doppler signals, or combinations thereof. In some embodiments, the ultrasound transducer may be coupled with a biopsy needle, and the processor may be further configured to generate an instruction for adjusting the ultrasound transducer to align the biopsy needle with the corrected biopsy path. In some examples, the plurality of tissue types may include various grades of cancerous tissue. In some embodiments, the target region may include a prostate gland. In some examples, the targeted biopsy sample may specify a maximum number of different tissue types, a maximum amount of a single tissue type, a particular tissue type, or combinations thereof. In some embodiments, the user input may embody a selection of a preset targeted biopsy sample option or a narrative description of the targeted biopsy sample. In some examples, the user interface may include a touch screen configured to receive the user input, and the user input may include movement of a virtual needle displayed on the touch screen. In some embodiments, the processor may be configured to generate and cause to be displayed a live ultrasound image acquired from the biopsy plane on the user interface. In some examples, the processor may be further configured to overlay the spatial distribution map on the live ultrasound image. In some embodiments, the neural network may be operatively associated with a training algorithm configured to receive an array of known inputs and known outputs, and the known inputs may include ultrasound image frames containing at least one tissue type and a histopathological classification associated with the at least one tissue type contained in the ultrasound image frames. In some examples, the ultrasound pulses may be transmitted at a frequency of about 5 to about 9 MHz. In some embodiments, the spatial distribution map may be generated using mpMRI data of the target region.

In accordance with some examples, a method of ultrasound imaging may involve acquiring echo signals responsive to ultrasound pulses transmitted along a biopsy plane within a target region; obtaining a time series of sequential data frames associated with the echo signals; applying a neural network to the time series of sequential data frames, in which the neural network determines spatial locations and identities of a plurality of tissue types in the sequential data frames; generating a spatial distribution map to be displayed on a user interface in communication with the processor, the spatial distribution map labeling the coordinates of the plurality of tissue types identified within the target region; receiving a user input, via the user interface, indicating a targeted biopsy sample; and generating a corrected biopsy path based on the targeted biopsy sample.

In some examples, the plurality of tissue types may include various grades of cancerous tissue. In some embodiments, methods may further involve applying a feasibility constraint against the corrected biopsy path, the feasibility constraint being based on physical limitations of a biopsy. In some embodiments, methods may further involve generating an instruction for adjusting an ultrasound transducer to align a biopsy needle with the corrected biopsy path. In some embodiments, methods may further involve overlaying the spatial distribution map on a live ultrasound image displayed on the user interface. In some examples, the corrected biopsy path may be generated by direct user interaction with the spatial distribution map displayed on the user interface. In some embodiments, the identities of a plurality of tissue types may be identified by recognizing ultrasound signatures unique to histopathological classifications of each of the plurality of tissue types.

### BRIEF DESCRIPTION OF THE DRAWINGS

FIG. 1 is a schematic illustration of a transrectal biopsy performed with an ultrasound probe and biopsy needle coupled thereto in accordance with principles of the present disclosure.

FIG. 2 is a schematic illustration of a transperineal biopsy performed with an ultrasound probe and a biopsy needle mounted on a template in accordance with principles of the present disclosure.

FIG. 3 is a block diagram of an ultrasound system in accordance with principles of the present disclosure.

FIG. 4 is a block diagram of another ultrasound system in accordance with principles of the present disclosure.

FIG. 5 is a schematic illustration of a tissue distribution map indicating various tissue types overlaid onto an ultrasound image in accordance with principles of the present disclosure.

FIG. 6 is a flow diagram of a method of ultrasound imaging performed in accordance with principles of the present disclosure.

### DETAILED DESCRIPTION

The following description of certain embodiments is merely exemplary in nature and is in no way intended to limit the invention or its applications or uses. In the following detailed description of embodiments of the present systems and methods, reference is made to the accompanying drawings which form a part hereof, and which are shown by way of illustration specific embodiments in which the described systems and methods may be practiced. These embodiments are described in sufficient detail to enable those skilled in the art to practice presently disclosed systems and methods, and it is to be understood that other embodiments may be utilized and that structural and logical changes may be made without departing from the spirit and scope of the present system. Moreover, for the purpose of clarity, detailed descriptions of certain features will not be discussed when they would be apparent to those with skill in the art so as not to obscure the description of the present system. The following detailed description is therefore not to be taken in a limiting sense, and the scope of the present system is defined only by the appended claims.

An ultrasound system according to the present disclosure may utilize a neural network, for example a deep neural network (DNN), a convolutional neural network (CNN) or the like, to identify and differentiate various tissue types, e.g., various grades of cancerous tissue, present within a target region subjected to ultrasound imaging. The neural network can further delineate distinct sub-regions of each tissue type identified along a biopsy plane. In some examples, the neural network may be trained using any of a variety of currently known or later developed machine learning techniques to obtain a neural network (e.g., a machine-trained algorithm or hardware-based system of nodes) that are able to analyze input data in the form of ultrasound image frames and associated histopathological classifications, and identify certain features therefrom, including the presence and spatial distribution of one or more tissue types or microstructures. Neural networks may provide an advantage over traditional forms of computer programming algorithms in that they can be generalized and trained to recognize data set features and their locations by analyzing data set samples rather than by reliance on specialized computer code. By presenting appropriate input and output data to a neural network training algorithm, the neural network of an ultrasound system according to the present disclosure can be trained to identify specific tissue types and the spatial locations of the identified tissue types within a biopsy plane in real time during an ultrasound scan, optionally producing a map of the target region that shows the tissue distribution. A processor communicatively coupled with the neural network can then determine a corrected biopsy path for an invasive object, e.g., needle. The corrected path can be configured to ensure the collection of the specific tissue type(s), e.g., specific cancer grades, prioritized by a user, e.g., a treating clinician. Determining the spatial distribution of specific grades of cancerous tissue within a target region using ultrasound and determining a corrected biopsy path based on the distribution information improves diagnostic precision and the treatment decisions based on the diagnoses.

An ultrasound system in accordance with principles of the present invention may include or be operatively coupled to an ultrasound transducer configured to transmit ultrasound pulses toward a medium, e.g., a human body or specific portions thereof, and generate echo signals responsive to the ultrasound pulses. The ultrasound system may include a beamformer configured to perform transmit and/or receive beamforming, and a display configured to display, in some examples, ultrasound images generated by the ultrasound imaging system. The ultrasound imaging system may include one or more processors and a neural network. The ultrasound system can be coupled with an mpMRI system, thereby enabling communication between the two components. The ultrasound system may also be coupled with a biopsy needle or biopsy gun needle configured to fire into a targeted tissue along a predetermined biopsy path.

The neural network implemented according to the present disclosure may be hardware—(e.g., neurons are represented by physical components) or software-based (e.g., neurons and pathways implemented in a software application), and can use a variety of topologies and learning algorithms for training the neural network to produce the desired output. For example, a software-based neural network may be implemented using a processor (e.g., single or multi-core CPU, a single GPU or GPU cluster, or multiple processors arranged for parallel-processing) configured to execute instructions, which may be stored in computer readable medium, and which when executed cause the processor to perform a machine-trained algorithm for identifying, delineating and/or labeling distinct tissue types imaged along a biopsy plane. The ultrasound system may include a display and/or graphics processor operable to display live ultrasound images and a tissue distribution map denoting various tissue types present within the images. Additional graphical information can also be displayed, which may include annotations, user instructions, tissue information, patient information, indicators, and other graphical components, in a display window for display on a user interface of the ultrasound system, which may be interactive, e.g., responsive to user touch. In some embodiments, the ultrasound images and tissue information, including information regarding cancerous tissue types and coordinates, may be provided to a storage and/or memory device, such as a picture archiving and communication system (PACS) for reporting purposes or future machine training (e.g., to continue to enhance the performance of the neural network). In some examples, ultrasound images obtained during a scan may not be displayed to the user operating the ultrasound system, but may be analyzed by the system for the presence, absence, and/or distribution of cancerous tissue in real time as an ultrasound scan is performed.

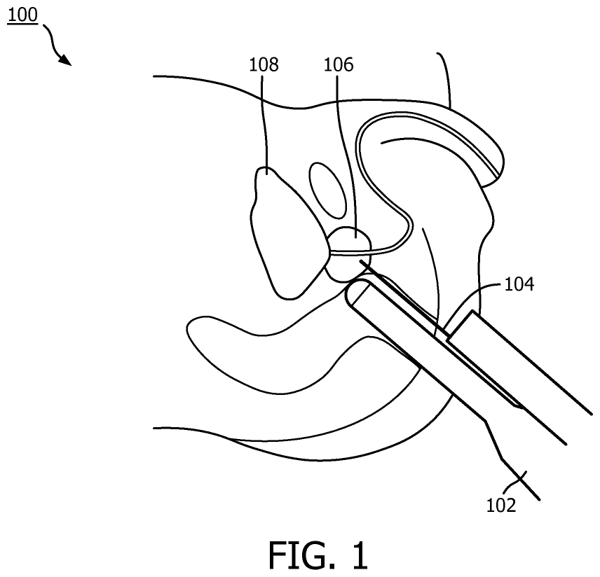

FIG. 1 shows an example of a transrectal biopsy procedure 100 performed according to principles of the present disclosure. The procedure 100, which may also be referred to as the “free-hand” transrectal biopsy, involves using an ultrasound probe 102 coupled with a biopsy needle 104, which can be mounted directly on the probe or on an adapter apparatus, e.g., needle guide, coupled with the probe in some examples. Together, the probe 102 and the needle 104 can be inserted into a patient's rectum until the distal ends of the two components are adjacent to the prostate gland 106 and bladder 108. In this position, the ultrasound probe 102 can transmit ultrasound pulses and acquire echo signals responsive to the pulses from the prostate gland 106, and the needle 104 can collect a tissue sample along a path dictated by the orientation of the probe. In accordance with the systems and methods disclosed herein, the projected biopsy path of the needle 104 can be adjusted based on the tissue information gathered via ultrasound imaging, thereby generating a corrected biopsy path distinct from the original biopsy path. For example, after and/or while receiving ultrasound data acquired by the probe 102, systems disclosed herein can determine and display the spatial distribution of various types of cancerous and benign tissue present within the prostate gland 106 along the biopsy plane imaged by the probe. The distribution information can then be used to determine a corrected biopsy path, which may be based at least in part on preferences specified by a user regarding specific tissue type(s) targeted for biopsy. The probe 102 and biopsy needle 104 can then be adjusted to align the needle with the corrected biopsy path, and the needle can be inserted into the prostate gland 106 along the path to collect a tissue sample for further analysis. While FIG. 1 shows a transrectal biopsy procedure, the systems and methods described herein are not limited to prostate imaging and can be implemented with respect to various tissue types and organs, e.g., breast, liver, kidney, etc.

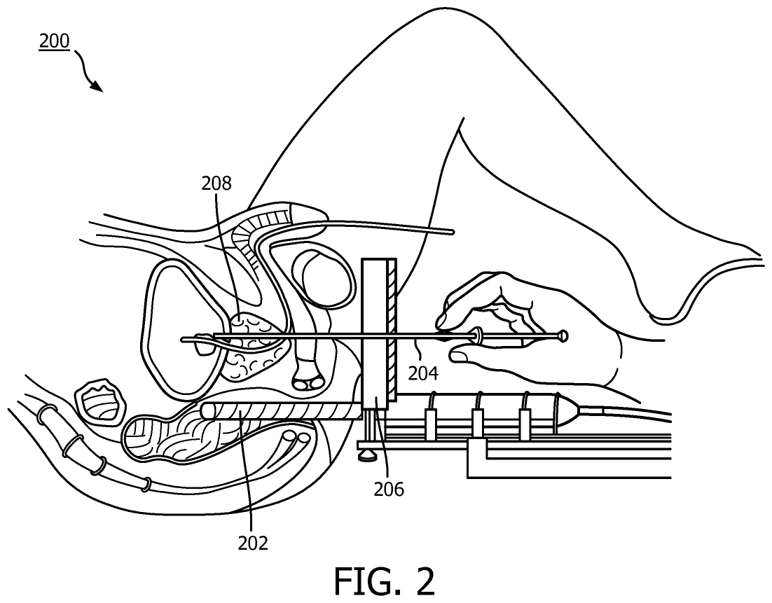

FIG. 2 shows an example of a transperineal biopsy procedure 200 performed according to principles of the present disclosure. As shown, the transperineal biopsy procedure 200 also involves the use of an ultrasound probe 202 and a biopsy needle 204. Unlike the transrectal biopsy procedure 100, the needle 204 used for transperineal biopsy is not mounted directly on the probe 202 or an adapter coupled with the probe. Instead, the needle 204 is selectively inserted into various slots defined by a template 206, such that the needle can be moved independently from the probe. During the procedure 200, the ultrasound probe 202 is inserted into a patient's rectum until a distal end of the probe is adjacent to prostate gland 208. Based on the ultrasound images collected using the probe 202, the systems disclosed herein can determine the spatial distribution of various cancerous and benign tissue types present within the prostate gland 208. A corrected biopsy path responsive to user preferences received by the system can be determined, which dictates the particular slot through which the needle 204 is inserted on the template 206. After aligning the needle 204 with the corrected biopsy path, the needle can be slid through the template 206, through the patient's perineum, and eventually into the prostate gland 208 along the biopsy path for tissue collection.

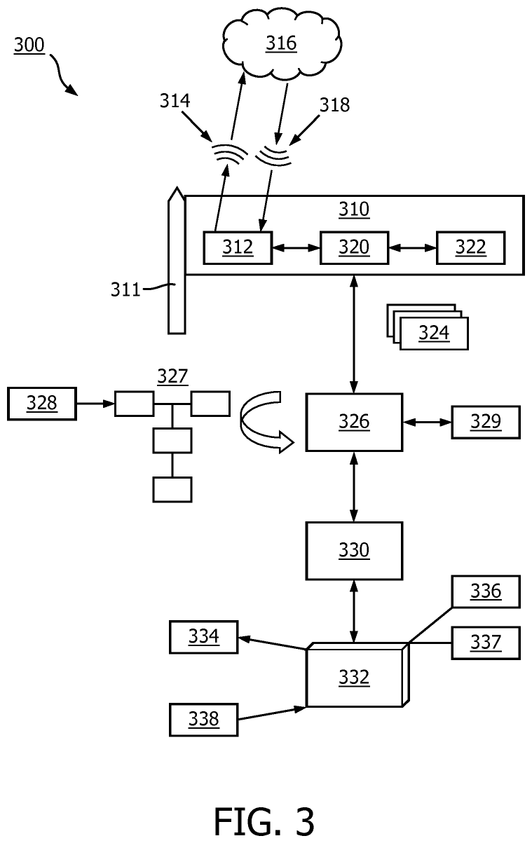

FIG. 3 shows an example ultrasound system 300 configured according to principles of the present disclosure. As shown, the system 300 can include an ultrasound data acquisition unit 310, which can be coupled with an invasive device 311, e.g., a biopsy needle, in some embodiments. The ultrasound data acquisition unit 310 can include an ultrasound transducer or probe comprising an ultrasound sensor array 312 configured to transmit ultrasound pulses 314 into a target region 316 of a subject, e.g., a prostate gland, and receive echoes 318 responsive to the transmitted pulses. In some examples, the ultrasound data acquisition unit 310 may also include a beamformer 320 and a signal processor 322, which may be configured to extract time series data embodying a plurality of ultrasound image frames 324 received sequentially at the array 312. To collect the time series data, a series of ultrasound image frames can be acquired from the same target region 316 over a period of time, e.g., less than 1 second up to about 2, about 4, about 6, about 8, about 16, about 24, about 48, or about 60 seconds. Various breath-holding and/or image registration techniques may be employed while imaging to compensate for movement and/or deformation of the target region 316 that may typically occur during normal breathing. One or more components of the data acquisition unit 310 can be varied or even omitted in different examples, and various types of ultrasound data may be collected. Using a continuous set of ultrasound data frames, time series data from the target region 316 can be generated, for example as described in U.S. Patent Application Publication No. 2010/0063393 A1, which is incorporated by reference in its entirety herein. In some examples, the data acquisition unit 310 may be configured to acquire radiofrequency (RF) data at a specific frame rate, e.g., about 5 to about 9 MHz. In additional examples, the data acquisition unit 310 may be configured to generate processed ultrasound data, e.g., B-mode, A-mode, M-mode-, Doppler, or 3D data. In some examples, the signal processor 322 may be housed with the sensor array 312 or it may be physically separate from but communicatively (e.g., via a wired or wireless connection) coupled thereto.

The system 300 can further include one or more processors communicatively coupled with the data acquisition unit 310. In some examples, the system can include a data processor 326, e.g., a computational module or circuitry (e.g., application specific integrated circuit (ASIC), configured to implement a neural network 327. The neural network 327 may be configured to receive the image frames 324, which may comprise a time series of sequential data frames 324 associated with the echo signals 318, and identify the tissue types, e.g., various grades of cancerous tissue or benign tissue, present within the image frames. The neural network 327 may also be configured to determine the spatial locations of the tissue types identified within the target region 316 and generate a tissue distribution map of the tissue types present within the imaged region.

To train the neural network 327, various types of training data 328 may be input into the network. The training data 328 may include image data embodying ultrasound signatures that correspond to specific tissue types, along with histopathological classifications of the specific tissue types. Through training, the neural network 327 can learn to associate certain ultrasound signatures with specific histopathological tissue classifications. The input data used for training can be gathered in various ways. For example, for each human subject included within a large patient population, time series ultrasound data can be collected from a particular target region, such as the prostate gland. A physical tissue sample of the imaged target region can also be collected from each subject, which can then be classified according to histopathological guidelines. Thus, two data sets can be collected for each subject in the patient population: a first data set containing time series ultrasound data of a target region, and a second data set containing histopathological classifications corresponding to each target region represented in the first data set. Accordingly, the ground truth, i.e., whether a given tissue region is cancerous or benign, for each sample represented in the patient population is known, along with the specific grade(s) of any cancerous tissue present within each sample. Grades of cancerous tissue may be based on the Gleason scoring system, which assigns numerical scores to tissue samples on a scale of 1 to 5, each number representative of cancer aggressiveness, e.g., low, medium or high. Lower Gleason scores typically indicate normal or slightly-abnormal tissue, while higher Gleason scores typically indicate abnormal and sometimes cancerous tissue.

Time and frequency domain analysis can be applied to the input training data 328 to extract representative features therefrom. Using the framework of the neural network 327, the extracted features, and the known ground truth of each tissue sample, a classifier layer within the network can be trained to separate and interpret tissue regions and identify cancer tissue grade based on the extracted features derived from ultrasound signals. In other words, the neural network 327 can learn what benign tissue ultrasound signals look like by processing a large number of ultrasound signatures gathered from benign tissue. Likewise, the neural network 327 can learn what cancerous tissue looks like by processing a large number of ultrasound signatures gathered from cancerous tissue.

After training the neural network 327 to distinguish benign tissue signatures from cancerous tissue signatures, and different cancerous tissue signatures from each other, the network may be configured to identify specific tissue types and their spatial coordinates along a biopsy plane within ultrasound data collected in real time. In specific examples, RF time series data can be generated during ultrasound imaging, the data embodying signals extracted from the echoes 318 received from the target region 316 by the data acquisition unit 310. The data can then be input into the trained neural network 327, which is configured to extract certain features from the data. The features can be examined by a classifier layer within the neural network 327, which is configured to identify tissue type(s), e.g., according to Gleason score, based on the extracted features. The tissue types identified can be mapped to spatial locations within the target region 316, and a map showing tissue type distribution can be output from the neural network 327. Outputs from the neural network 327 regarding tissue distribution can be fused with mpMRI data to generate the tissue type distribution map. In some embodiments, the data processor 326 can be communicatively coupled with an mpMRI system 329, which may be configured to perform mpMRI and/or store pre-operative mpMRI data corresponding to the target region 316 imaged by the ultrasound data acquisition unit 310. Examples of mpMRI systems compatible with the ultrasound imaging system 300 shown in FIG. 3 include UroNav by Philips Koninklijke Philips N. V. (“Philips”). Philips UroNav is a targeted biopsy platform for prostate cancer equipped with multi-modal fusion capability. The data processor 326 may be configured to fuse the mpMRI data with the ultrasound image data before or after application of the neural network 327.

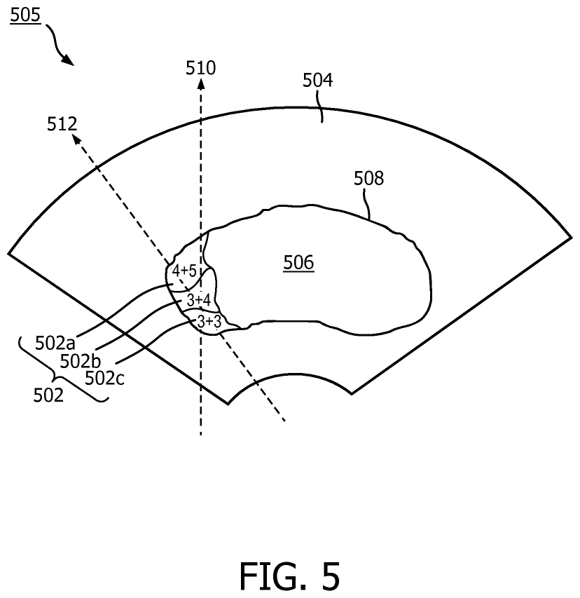

The tissue distribution data output by the neural network 327 can be used by the data processor 326, or one more additional or alternative processors, to determine a corrected biopsy path. The configuration of the corrected biopsy path can vary depending on the preferences of a user and in some cases, the corrected biopsy path can be determined automatically, without user input. Automatic biopsy path correction can operate to generate a path that results in a biopsy of the greatest tissue type diversity, e.g., maximizing the number of different cancer grades, present within the target region. Additional examples of biopsy path correction customization are detailed below in connection with FIG. 5.

As further shown in FIG. 3, the system 300 can also include a display processor 330 coupled with the data processor 326 and a user interface 332. In some examples, the display processor 330 can be configured to generate live ultrasound images 334 form the image frames 324 and a tissue distribution map 336. The tissue distribution map 336 may include an indication of a location of an original biopsy path, which may be based on the angle and orientation of the ultrasound transducer performing the ultrasound imaging. The tissue distribution map 336 may also include the corrected biopsy path determined by the system 300. In addition, the user interface 332 may also be configured to display one or more messages 337, which may include instructions for adjusting the ultrasound transducer 312 in the manner necessary to align a biopsy needle 311 coupled thereto with the corrected biopsy path. In some examples, the messages 337 may include an alert, which may convey to the user that a corrected biopsy path consistent with the user's preferences cannot be feasibly attained. The user interface 332 may also be configured to receive a user input 338 at any time before, during, or after an ultrasound scan. In some examples, the user input 338 can include a selection of a preset path correction option specifying tissue types to be obtained along a corrected biopsy path. Example preset selections may embody instructions to “maximize tissue diversity,” “maximize grade 4+5 tissue,” or “maximize cancerous tissue.” In additional examples, the user input 338 can include ad hoc preferences input by a user. According to such examples, the system 300 may be include a natural language processor configured to parse and/or interpret the text inputted by the user.

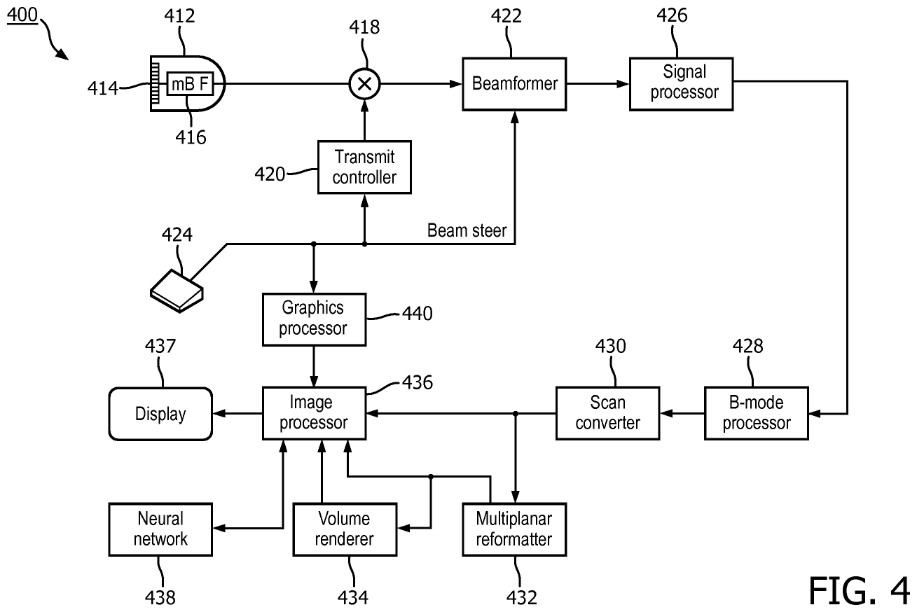

FIG. 4 is a block diagram of another ultrasound system in accordance with principles of the present disclosure. One or more components shown in FIG. 4 may be included within a system configured to identify specific tissue types present along a biopsy plane of a target region, determine the spatial distribution of the identified tissue types, generate a tissue distribution map depicting the spatial distribution, and/or determine a corrected biopsy path configured to sample the tissues identified in the target region in accordance with user preferences. For example, any of the above-described functions of the signal processor 322 or data processor 326 may be implemented and/or controlled by one or more of the processing components shown in FIG. 4, including for example, signal processor 426, B-mode processor 428, scan converter 430, multiplanar reformatter 423, volume renderer 434 and/or image processor 436.

In the ultrasonic imaging system of FIG. 4, an ultrasound probe 412 includes a transducer array 414 for transmitting ultrasonic waves into a region containing a feature, e.g., a prostate gland or other organ, and receiving echo information responsive to the transmitted waves. In various embodiments, the transducer array 414 may be a matrix array or a one-dimensional linear array. The transducer array may be coupled to a microbeamformer 416 in the probe 412 which may control the transmission and reception of signals by the transducer elements in the array such that time series data is collected by the probe 412. In the example shown, the microbeamformer 416 is coupled by the probe cable to a transmit/receive (T/R) switch 418, which switches between transmission and reception and protects the main beamformer 422 from high energy transmit signals. In some embodiments, the T/R switch 418 and other elements in the system can be included in the transducer probe rather than in a separate ultrasound system component. The transmission of ultrasonic beams from the transducer array 414 under control of the microbeamformer 416 may be directed by the transmit controller 420 coupled to the T/R switch 418 and the beamformer 422, which receives input, e.g., from the user's operation of the user interface or control panel 424. A function that may be controlled by the transmit controller 420 is the direction in which beams are steered. Beams may be steered straight ahead from (orthogonal to) the transducer array, or at different angles for a wider field of view. The partially beamformed signals produced by the microbeamformer 416 are coupled to a main beamformer 422 where partially beamformed signals from individual patches of transducer elements are combined into a fully beamformed signal.

The beamformed signals may be communicated to a signal processor 426. The signal processor 426 may process the received echo signals in various ways, such as bandpass filtering, decimation, I and Q component separation, and/or harmonic signal separation. The signal processor 426 may also perform additional signal enhancement via speckle reduction, signal compounding, and/or noise elimination. In some examples, data generated by the different processing techniques employed by the signal processor 426 may be used by a neural network to identify distinct tissue types indicated by unique ultrasound signals embodied within the ultrasound data. The processed signals may be coupled to a B-mode processor 428 in some examples. The signals produced by the B-mode processor 428 may be coupled to a scan converter 430 and a multiplanar reformatter 432. The scan converter 430 may arrange the echo signals in the spatial relationship from which they were received in a desired image format. For instance, the scan converter 430 may arrange the echo signals into a two dimensional (2D) sector-shaped format. The multiplanar reformatter 432 may convert echoes which are received from points in a common plane in a volumetric region of the body into an ultrasonic image of that plane, as described in U.S. Pat. No. 6,443,896 (Detmer). In some examples, a volume renderer 434 may convert the echo signals of a 3D data set into a projected 3D image as viewed from a given reference point, e.g., as described in U.S. Pat. No. 6,530,885 (Entrekin et al.). The 2D or 3D images may be communicated from the scan converter 430, multiplanar reformatter 432, and volume renderer 434 to an image processor 436 for further enhancement, buffering and/or temporary storage for display on an image display 437. Prior to their display, a neural network 438 may be implemented to identify tissue types present within a target region imaged by the probe 412 and delineate the spatial distribution of such tissue types. The neural network 438 may also be configured to produce a tissue distribution map based on the identification and spatial delineation performed. In embodiments, the neural network 438 may be implemented at various processing stages, e.g., prior to the processing performed by the image processor 436, volume renderer 434, multiplanar reformatter 432, and/or scan converter 430. In specific examples, the neural network 438 can be applied to raw RF data, i.e., without processing performed by the B-mode processor 428. A graphics processor 440 can generate graphic overlays for display with the ultrasound images. These graphic overlays may contain, e.g., standard identifying information such as patient name, date and time of the image, imaging parameters, and the like, and also various outputs generated by the neural network 438, such as the tissue distribution map, an original biopsy path, a corrected biopsy path, messages directed toward a user, and/or instructions for adjusting the ultrasound probe 412 and/or a biopsy needle used in tandem with the probe during a biopsy procedure. In some examples, the graphics processor 440 may receive input from the user interface 424, such as a typed patient name or confirmation that an instruction displayed or emitted from the interface has been acknowledged by the user of the system 400. The user interface 424 may also receive input embodying user preferences for the selection of specifically targeted tissue types. Input received at the user interface can be compared to the tissue distribution map generated by the neural network and ultimately used to determine a corrected biopsy path consistent with the selection. The user interface may also be coupled to the multiplanar reformatter 432 for selection and control of a display of multiple multiplanar reformatted (MPR) images.

FIG. 5 is a schematic illustration of a tissue distribution map 502 overlaid onto an ultrasound image 504 displayed on an interactive user interface 505 in accordance with principles of the present disclosure. The tissue distribution map 502, generated by a neural network described herein, may highlight a plurality of distinct tissue sub-regions 502a, 502b, 502c. As shown, the map 502 may be confined within an organ 506. The boundary 508 of the organ can be derived by mpMRI data collected offline, e.g., prior to ultrasound imaging and biopsy, and fused with ultrasound imaging data. An original biopsy path 510 is shown, along with a corrected biopsy path 512.

Each sub-region 502a, 502b, 502c contains a distinct tissue type, as determined in accordance with the Gleason scoring system in this particular embodiment. In particular, the first sub-region 502a contains tissue having a Gleason score of 4+5, while the second sub-region 502b contains tissue having a score of 3+4, and the third sub-region 502c contains tissue having a Gleason score of 3+3. Thus, the first sub-region 502a contains tissue exhibiting the most aggressive growth, making this tissue the most likely to be cancerous. The original biopsy path 510 passes through each of the sub-regions 502a, 502b, 502c delineated in the map 502; however, not every sub-region is sampled equally. The first sub-region 502a, for example, is only tangentially intersected by the original biopsy path 510. Especially because the first sub-region 502a harbors the most aggressive tissue, a user may elect to modify the original biopsy path 510 to arrive at the corrected biopsy path 512. As is clear from the map 502, the corrected biopsy path 512 passes directly through each sub-region 502a, 502b, 502c, thereby increasing the likelihood that adequate tissue samples will be collected therefrom.

The corrected biopsy path 512 may be determined in various ways, which may depend at least in part on the preferences input by a user, who may prioritize certain tissue types over others in view of clinical objectives. For example, a user can specify that a certain cancer grade, e.g., 4+5, should be biopsied, irrespective of the other cancerous tissue grades that may be present with a target region along the imaged biopsy plane. Such preferences can be received at the user interface 505 and used to determine a corrected biopsy path consistent with the preferences. In some embodiments, the preferences may be stored as preset options selectable by a user. Preset options may include instructions for the system to determine a corrected biopsy path configured to collect a specific ratio of different tissue types, or to collect tissue types in compliance with particular clinical guidelines. For instance, a user may specify that the corrected biopsy path must be configured to obtain 50% of the tissue sample from the first sub-region 502a, 30% of the tissue sample from the second sub-region 502b, and 20% of the tissue sample from the third sub-region 502c. As mentioned above, user preferences can also be received in ad-hoc fashion, e.g., via narrative descriptions of the targeted tissue type(s). Whether embodied in preset selections or ad hoc descriptions, the user preferences may be customized in the manner necessary to obtain a biopsy sample sufficient to make an accurate clinical diagnosis for a specific patient. The user can customize the path correction preferences at various times. In some embodiments, the user can enter the preferences in advance of an ultrasound scan. In some examples, a user can modify the preferences after tissue type distribution information is obtained. In addition or alternatively, a user can directly specify a corrected biopsy path by interacting directly with the tissue distribution map 502 via the user interface 505. According to such examples, a user may click (or simply touch if the user interface comprises a touch screen) a needle, line, or icon representing the original biopsy path and drag it to a second, corrected location on the user interface. In some examples, the user interface 505 can be configured such that the user can select to operate the ultrasound system in “learning mode,” during which the system automatically adapts to user input responsive to the spatial distribution data output by the neural network and displayed on the user interface. In addition, the corrected biopsy path 512 may automatically correct for any misalignment between pre-biopsy mpMRI locations and spatial coordinates determined in real-time via ultrasound.

Pursuant to determining the corrected biopsy path 512 that satisfies the specified user preferences, the system can apply a “most-feasible” constraint, which may comprise a geometric constraint that limits the number of corrected biopsy paths that are actually practical given the set-up of the biopsy procedure. For example, applying the most-feasible constraint may eliminate corrected biopsy paths that are not physically possible based on the biopsy collection angle required to obtain samples along such certain paths. The most feasible constraint may be applied after one or more corrected biopsy paths 512 are determined, but optionally before such paths are displayed on the user interface 505. The system may be further configured to communicate an alert when the most-feasible constraint impacts the corrected path results. In some examples, multiple corrected biopsy paths 512 may be displayed that are configured to satisfy, in combination, the preferences received from the user. Multi-path determinations may be automatically generated and displayed when it has been determined that the most-feasible constraint impacts the results and/or when satisfaction of the received user preferences is not possible along any one given biopsy path.

The configuration of the tissue distribution map 502 can vary. In some embodiments, the map 502 can comprise a color map configured to label different tissue types with different colors. For example, benign tissue can be indicated in blue, while cancerous tissue having high Gleason scores can be indicated in red or orange. In addition or alternatively, the map 502 can be configured to superimpose Gleason scores directly onto corresponding tissue sub-regions, as shown. In some examples, the user interface may also be configured to show various statistics derived from the color map and the biopsy path(s) displayed thereon. For example, the user interface can show the percentage of coverage for each tissue grade included in a given biopsy path. The user interface can show the spatial coordinates and boundaries of all tissue types identified by the neural network.

The user interface 505 can be configured to display an instruction for adjusting an ultrasound probe and/or biopsy needle, depending on whether a free-hand or transperineal biopsy is being performed, in the manner necessary to align the probe/needle with the corrected biopsy path 512. For example, the user interface 505 can display instructions that read “tilt laterally,” “tilt dorsally,” or “rotate 90 degrees,” for example. The instructions can be conveyed according to various modes of communication. In some examples, the instructions may be displayed in text format, while in other examples the instructions may be communicated in audio format, or using symbols, graphics, etc. In additional embodiments, the instructions can be communicated with a mechanism configured to adjust the ultrasound probe and/or biopsy needle without manual intervention, e.g., using a robotic armature coupled with the probe and/or biopsy needle. Examples may also involve automatic adjustment of one or more ultrasound imaging modalities, e.g., beam angle, focal depth, acquisition frame rate, etc.

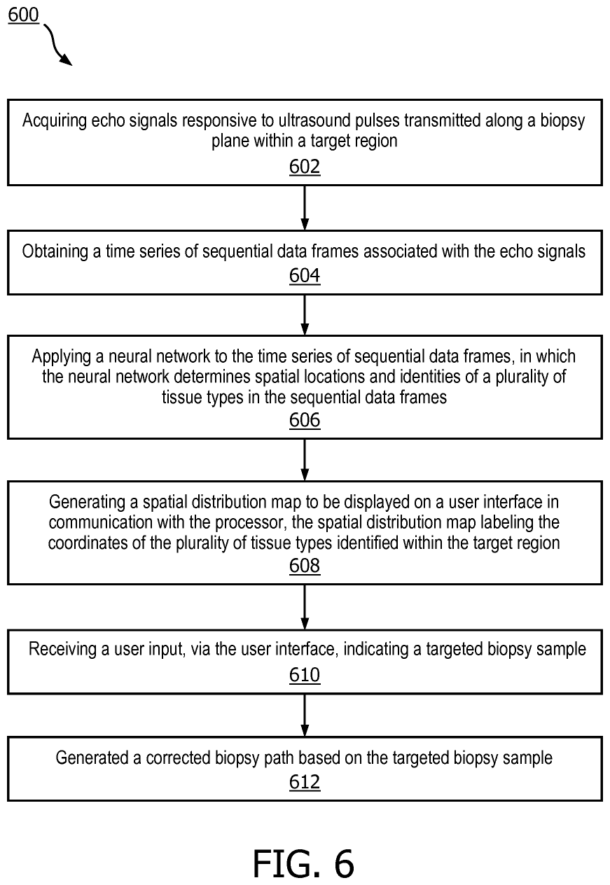

FIG. 6 is a flow diagram of a method of ultrasound imaging performed in accordance with principles of the present disclosure. The example method 600 shows the steps that may be utilized, in any sequence, by the ultrasound systems and/or apparatuses described herein for delineating tissue types and spatial locations along a biopsy plane, generating a spatial distribution map, and determining a corrected biopsy path.

In the embodiment shown, the method begins at block 602 by “acquiring echo signals responsive to ultrasound pulses transmitted along a biopsy plane within a target region.” Depending on the biopsy being performed, the target region may vary. In some examples, the target region can include the prostate gland. Various types of ultrasound transducers can be employed to acquire the echo signals. The transducers can be configured specifically to accommodate different bodily features. For example, a transrectal ultrasound probe may be used.

At block 604, the method involves “obtaining a time series of sequential data frames associated with the echo signals.” The time series of sequential data frames can embody radio frequency signals, B-mode signals, Doppler signals, or combinations thereof.

At block 606, the method involves “applying a neural network to the time series of sequential data frames, in which the neural network determines spatial locations and identities of a plurality of tissue types in the sequential data frames.” In some examples, the plurality of tissue types may include various grades of cancerous tissue, e.g., moderately aggressive, highly aggressive, or slightly abnormal. In some examples, cancerous tissue grades may be defined according to Gleason score on a numerical scale ranging from 1 to 5. In various embodiments, the tissue types can be identified by recognizing ultrasound signatures unique to histopathological classifications of each tissue type.

At block 608, the method involves “generating a spatial distribution map to be displayed on a user interface in communication with the processor, the spatial distribution map labeling the coordinates of the plurality of tissue types identified within the target region.” The spatial distribution map can be overlaid on a live ultrasound image displayed on a user interface in some embodiments. In addition or alternatively, the spatial distribution map can be a color map.

At block 610, the method involves “receiving a user input, via the user interface, indicating a targeted biopsy sample.” The targeted biopsy sample can specify a maximum number of different tissue types, a maximum amount of a single tissue type and/or a particular tissue type to be sampled, according to user preferences.

At block 612, the method involves “generating a corrected biopsy path based on the targeted biopsy sample.” The corrected biopsy path can be generated by direct user interaction with the spatial distribution map displayed on the user interface. Additional factors can also impact the corrected biopsy path. For example, the method may further involve applying a feasibility constraint against the corrected biopsy path. The feasibility constraint may be based on physical limitations of the biopsy procedure being performed. Physical limitations may relate to the practicality of positioning the biopsy needle at certain angles, for example. Internal bodily structures, along with the shape and size of the ultrasound transducer apparatus may each impact the feasibility constraint. Embodiments may also involve generating an instruction for adjusting the ultrasound transducer in the manner necessary to align a biopsy needle with the corrected biopsy path, to the extent such alignment is possible in view of the feasibility constraint.

In various embodiments where components, systems and/or methods are implemented using a programmable device, such as a computer-based system or programmable logic, it should be appreciated that the above-described systems and methods can be implemented using any of various known or later developed programming languages, such as “C”, “C++”, “FORTRAN”, “Pascal”, “VHDL” and the like. Accordingly, various storage media, such as magnetic computer disks, optical disks, electronic memories and the like, can be prepared that can contain information that can direct a device, such as a computer, to implement the above-described systems and/or methods. Once an appropriate device has access to the information and programs contained on the storage media, the storage media can provide the information and programs to the device, thus enabling the device to perform functions of the systems and/or methods described herein. For example, if a computer disk containing appropriate materials, such as a source file, an object file, an executable file or the like, were provided to a computer, the computer could receive the information, appropriately configure itself and perform the functions of the various systems and methods outlined in the diagrams and flowcharts above to implement the various functions. That is, the computer could receive various portions of information from the disk relating to different elements of the above-described systems and/or methods, implement the individual systems and/or methods and coordinate the functions of the individual systems and/or methods described above.

In view of this disclosure it is noted that the various methods and devices described herein can be implemented in hardware, software and firmware. Further, the various methods and parameters are included by way of example only and not in any limiting sense. In view of this disclosure, those of ordinary skill in the art can implement the present teachings in determining their own techniques and needed equipment to affect these techniques, while remaining within the scope of the invention. The functionality of one or more of the processors described herein may be incorporated into a fewer number or a single processing unit (e.g., a CPU) and may be implemented using application specific integrated circuits (ASICs) or general purpose processing circuits which are programmed responsive to executable instruction to perform the functions described herein.

Although the present system may have been described with particular reference to an ultrasound imaging system, it is also envisioned that the present system can be extended to other medical imaging systems where one or more images are obtained in a systematic manner. Accordingly, the present system may be used to obtain and/or record image information related to, but not limited to renal, testicular, breast, ovarian, uterine, thyroid, hepatic, lung, musculoskeletal, splenic, cardiac, arterial and vascular systems, as well as other imaging applications related to ultrasound-guided interventions. Further, the present system may also include one or more programs which may be used with conventional imaging systems so that they may provide features and advantages of the present system. Certain additional advantages and features of this disclosure may be apparent to those skilled in the art upon studying the disclosure, or may be experienced by persons employing the novel system and method of the present disclosure. Another advantage of the present systems and method may be that conventional medical image systems can be easily upgraded to incorporate the features and advantages of the present systems, devices, and methods.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

Finally, the above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

What is claimed is:

1. An ultrasound imaging system comprising:
an ultrasound transducer configured to acquire echo signals responsive to ultrasound pulses transmitted along a biopsy plane within a target region;
a processor in communication with the ultrasound transducer and configured to:
obtain a time series of sequential data frames associated with the echo signals;
apply a neural network to the time series of sequential data frames, in which the neural network determines spatial locations and identities of a plurality of tissue types in the sequential data frames;
generate a spatial distribution map to be displayed on a user interface in communication with the processor, the spatial distribution map labeling the coordinates of the plurality of tissue types identified within the target region;
receive a user input, via the user interface, indicating a targeted biopsy sample; and
generate a corrected biopsy path based on the targeted biopsy sample.

2. The ultrasound imaging system of claim 1, wherein the time series of sequential data frames embody radio frequency signals, B-mode signals, Doppler signals, or combinations thereof.

3. The ultrasound imaging system of claim 1, wherein the ultrasound transducer is coupled with a biopsy needle, and the processor is further configured to generate an instruction for adjusting the ultrasound transducer to align the biopsy needle with the corrected biopsy path.

4. The ultrasound imaging system of claim 1, wherein the plurality of tissue types comprise various grades of cancerous tissue.

5. The ultrasound imaging system of claim 1, wherein the target region comprises a prostate gland.

6. The ultrasound imaging system of claim 1, wherein the targeted biopsy sample comprises a maximum number of different tissue types, a maximum amount of a single tissue type, a particular tissue type, or combinations thereof.

7. The ultrasound imaging system of claim 1, wherein the user input comprises a selection of a preset targeted biopsy sample option or a narrative description of the targeted biopsy sample.

8. The ultrasound imaging system of claim 1, wherein the user interface comprises a touch screen configured to receive the user input, and wherein the user input comprises movement of a virtual needle displayed on the touch screen.

9. The ultrasound imaging system of claim 1, wherein the processor is configured to generate and cause to be displayed a live ultrasound image acquired from the biopsy plane on the user interface.

10. The ultrasound imaging system of claim 9, wherein the processor is further configured to overlay the spatial distribution map on the live ultrasound image.

11. The ultrasound imaging system of claim 1, wherein the neural network is operatively associated with a training algorithm configured to receive an array of known inputs and known outputs, wherein the known inputs comprise ultrasound image frames containing at least one tissue type and a histopathological classification associated with the at least one tissue type contained in the ultrasound image frames.

12. The ultrasound imaging system of claim 1, wherein the ultrasound pulses are transmitted at a frequency of about 5 to about 9 MHz.

13. The ultrasound imaging system of claim 1, wherein the spatial distribution map is generated using mpMRI data of the target region.

14. A method of ultrasound imaging, the method comprising:
acquiring echo signals responsive to ultrasound pulses transmitted along a biopsy plane within a target region;
obtaining a time series of sequential data frames associated with the echo signals;
applying a neural network to the time series of sequential data frames, in which the neural network determines spatial locations and identities of a plurality of tissue types in the sequential data frames;
generating a spatial distribution map to be displayed on a user interface in communication with the processor, the spatial distribution map labeling the coordinates of the plurality of tissue types identified within the target region;
receiving a user input, via the user interface, indicating a targeted biopsy sample; and
generating a corrected biopsy path based on the targeted biopsy sample.

15. The method of claim 14, wherein the plurality of tissue types comprise various grades of cancerous tissue.

16. The method of claim 14, further comprising applying a feasibility constraint against the corrected biopsy path, wherein the feasibility constraint is based on physical limitations of a biopsy.

17. The method of claim 14, further comprising generating an instruction for adjusting an ultrasound transducer to align a biopsy needle with the corrected biopsy path.

18. The method of claim 14, further comprising overlaying the spatial distribution map on a live ultrasound image displayed on the user interface.

19. The method of claim 14, wherein the corrected biopsy path is generated by direct user interaction with the spatial distribution map displayed on the user interface.

20. The method of claim 14, wherein the identities of a plurality of tissue types are identified by recognizing ultrasound signatures unique to histopathological classifications of each of the plurality of tissue types.
